(19) United States
(12) Patent Application Publication
(10) Pub. No.: US 2014/0018386 A1
(43) Pub. Date: Jan. 16, 2014

(54) LAQUINIMOD FORMULATIONS WITHOUT ALKALIZING AGENT

(71) Applicants: Gadi Sarfati, Kibbutz Beit Guvrin (IL); Ioana Lovinger, Kfar Saba (IL); Danit Licht, Givat Shmuel (IL); Muhammad Safadi, Nazareth (IL)

(72) Inventors: Gadi Sarfati, Kibbutz Beit Guvrin (IL); Ioana Lovinger, Kfar Saba (IL); Danit Licht, Givat Shmuel (IL); Muhammad Safadi, Nazareth (IL)

(73) Assignee: Teva Pharmaceutical Industries, Ltd., Petach-Tikva (IL)

(21) Appl. No.: 13/938,733

(22) Filed: Jul. 10, 2013

Related U.S. Application Data
(60) Provisional application No. 61/670,268, filed on Jul. 11, 2012.

(51) Int. Cl. A61K 31/4704

(52) U.S. Cl. CPC A61K 31/4704
USPC 514/312

(57) ABSTRACT

The subject invention provides a stable pharmaceutical composition comprising a therapeutically effective amount of laquinimod, an amount of a filler, and an amount of a lubricant, wherein the stable pharmaceutical composition is free of an alkalizing agent or an oxidation reducing agent. Also provided are processes for making the stable pharmaceutical composition and sealed packages comprising the stable pharmaceutical composition. Also provided is a method for treating a subject afflicted with a form of multiple sclerosis (MS) or for alleviating a symptom of MS in a subject afflicted with a form of MS comprising administering to the subject a stable pharmaceutical composition as described herein. Also provided is use of a stable pharmaceutical composition as described herein for treating a subject afflicted with a form of MS or for alleviating a symptom of MS in a subject afflicted with a form of multiple MS.

This application claims benefit of U.S. Provisional Application No. 61/670,268, filed Jul. 11, 2012, the entire content of which is hereby incorporated by reference herein.

Throughout this application various publications, published patent applications, and patents are referenced. The disclosures of these documents in their entireties are hereby incorporated by reference into this application in order to more fully describe the state of the art to which this invention pertains.

### BACKGROUND

Laquinimod is a compound which has been shown to be effective in the acute experimental autoimmune encephalomyelitis (aEAE) model (U.S. Pat. No. 6,077,851). Its chemical name is N-ethyl-N-phenyl-1,2-dihydro-4-hydroxy-5-chloro-1-methyl-2-oxoquinoline-3-carboxamide, and its Chemical Registry number is 248281-84-7. The processes of synthesis of laquinimod and the preparation of its sodium salt are disclosed in U.S. Pat. No. 6,077,851. An additional process of synthesis of laquinimod is disclosed in U.S. Pat. No. 6,875,869.

Pharmaceutical compositions comprising laquinimod sodium are disclosed in, e.g., U.S. Pat. No. 7,989,473 and PCT International Application Publication No. WO 2005/074899.

Laquinimod sodium has high oral bioavailability and has been suggested as an oral formulation for the treatment of Multiple Sclerosis (MS). (Polman, 2005 and Sandberg-Wollheim, 2005). Studies have also shown that laquinimod can reduce development of active MRI lesions in relapsing MS. (Polman 2005).

### SUMMARY OF THE INVENTION

The subject invention provides a stable pharmaceutical composition comprising a therapeutically effective amount of laquinimod, an amount of a filler, and an amount of a lubricant, wherein the stable pharmaceutical composition is free of an alkalizing agent or an oxidation reducing agent.

The subject invention also provides a process for making a stable pharmaceutical composition comprising a therapeutically effective amount of laquinimod, an amount of a filler and an amount of a lubricant, wherein the pharmaceutical composition is free of an alkalizing agent or an oxidation reducing agent, said process comprising: a) obtaining the laquinimod, the lubricant and the filler; b) mixing the laquinimod, the lubricant and the filler from step a) to achieve a dry mix free of an alkalizing agent or an oxidation reducing agent; and c) compressing the dry mix of step b) to form a tablet.

The subject invention also provides a process for making a stable pharmaceutical composition comprising a therapeutically effective amount of laquinimod, an amount of a filler and an amount of a lubricant, wherein the pharmaceutical composition is free of an alkalizing agent or an oxidation reducing agent, said process comprising: a) obtaining the laquinimod, the lubricant and the filler; b) adding the filler to a mixer; c) dissolving laquinimod in water to form a laquinimod solution; d) adding the laquinimod solution of step c) to the mixer of step b); e) mixing the laquinimod solution and the mannitol to form a granulate; f) drying the granulate from step e) to form a dried granulate; g) screening the dried granulate of step f); h) milling the granulate resulting from step g) to form a milled granulate; i) adding the lubricant to the milled granulate of step h) to form a mixture; j) blending the mixture of step i) into a mixer to achieve a dry mix free of an alkalizing agent or an oxidation reducing agent; and k) filling the dry mix of step j) into a capsule or compressing the dry mix of step j) to form a tablet.

The subject invention also provides a stable pharmaceutical composition comprising a therapeutically effective amount of laquinimod, an amount of a filler and an amount of a lubricant wherein the pharmaceutical composition is free of an alkalizing agent or an oxidation reducing agent, prepared by the processes described herein.

The subject invention also provides a sealed package comprising the stable pharmaceutical compositions described herein.

The subject invention also provides a sealed package containing a stable pharmaceutical composition comprising a therapeutically effective amount of laquinimod, an amount of a filler and an amount of a lubricant, wherein the pharmaceutical composition is free of an alkalizing agent or an oxidation reducing agent, and wherein the sealed package has a moisture permeability of not more than 9.2 mg/day per liter.

The subject invention also provides a method for treating a subject afflicted with a form of multiple sclerosis comprising administering to the subject a stable pharmaceutical composition as described herein so as to thereby treat the subject.

The subject invention also provides a method for alleviating a symptom of multiple sclerosis in a subject afflicted with a form of multiple sclerosis comprising administering to the subject a stable pharmaceutical composition as described herein so as to thereby alleviate the symptom of multiple sclerosis in the subject.

The subject invention also provides for use of a stable pharmaceutical composition as described herein for treating a subject afflicted with a form of multiple sclerosis.

The subject invention also provides for use of a stable pharmaceutical composition as described herein for alleviating a symptom of multiple sclerosis in a subject afflicted with a form of multiple sclerosis.

### DETAILED DESCRIPTION OF THE INVENTION

Laquinimod is a small molecule having the following chemical structure:

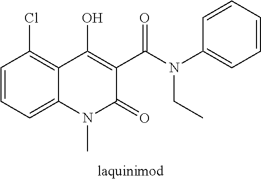 [SMILES: CCN(C(=O)c1c(O)c2c(Cl)cccc2n(C)c1=O)c1ccccc1]

It is an oral immunemodulator which has demonstrated therapeutic effect in various experimental inflammatory/autoimmune animal models, such as Experimental Autoimmune Encephalomyelitis (EAE), an animal model for Multiple Sclerosis (MS), Dextran Sodium Solphate (DSS) induced colitis for Inflammatory Bowel Disease, Non-Obese Diabetic (NOD) mice for Type I Diabetes (IDDM), Experimental Autoimmune Neuritis (EAN) for Guillain-Barre Syndrome, Systemic Lupus Erythematosus (SLE), lupus nephritis, lupus arthritis, Crohn's Disease and Rheumatoid arthritis. The therapeutic activity of laquinimod in these models results from a variety of mechanistic effects, including reduction of leukocyte infiltration into target tissues by modulation of chemokine-mediated T-cell adhesion, modulation of cytokine balance, down regulation of MHC class II resulting in alteration of antigen presentation, and effects on dendritic cells subpopulations.

The inventors have surprisingly found laquinimod formulations which are stable without alkalizing agents. Prior to this invention, it was thought in the art that alkalizing agents were necessary to provide stable laquinimod formulations.

### Embodiments

The subject invention provides a stable pharmaceutical composition comprising a therapeutically effective amount of laquinimod, an amount of a filler, and an amount of a lubricant, wherein the stable pharmaceutical composition is free of an alkalizing agent or an oxidation reducing agent.

In an embodiment of the present invention, the stable pharmaceutical composition is in a solid form composition. In another embodiment, the stable pharmaceutical composition is free of an alkalizing agent and free of an oxidation reducing agent.

In one embodiment, the moisture content of the stable pharmaceutical composition is no more than 4%. In another embodiment, the stable pharmaceutical composition contains less than 1.5% wt H₂O. In another embodiment, the stable pharmaceutical composition contains less than 0.5% wt H₂O. In yet another embodiment, the total amount of non-polar impurities in the composition is less than 0.5 wt % relative to the amount of laquinimod.

In one embodiment, the filler is present in the composition as solid particles. In another embodiment, the filler is lactose, lactose monohydrate, starch, isomalt, mannitol, sodium starch glycolate, sorbitol, lactose spray dried, lactose anhydrous, or a combination thereof. In yet another embodiment, the filler is mannitol or lactose monohydrate.

In one embodiment, the lubricant is present in the composition as solid particles. In another embodiment, the lubricant is magnesium stearate or sodium stearyl fumarate.

In one embodiment, the stable pharmaceutical composition is free of disintegrant. In another embodiment, the stable pharmaceutical composition is free of croscarmellose sodium.

In one embodiment, laquinimod is a pharmaceutically acceptable salt of laquinimod, which pharmaceutically acceptable salt is lithium salt, sodium salt or calcium salt. In another embodiment, the pharmaceutically acceptable salt of laquinimod is laquinimod sodium.

In one embodiment, laquinimod is present in the composition as solid particles.

In one embodiment, the therapeutically effective amount of laquinimod is 0.25 mg-1.5 mg. In another embodiment, the therapeutically effective amount of laquinimod is 0.5 mg. In another embodiment, the therapeutically effective amount of laquinimod is 0.6 mg. In another embodiment, the therapeutically effective amount of laquinimod is 1.0 mg. In yet another embodiment, the therapeutically effective amount of laquinimod is 1.2 mg.

In one embodiment, the lubricant is between 0.5-2.0% of the total weight of the stable pharmaceutical composition. In another embodiment, the filler is between 89.0-99.5% of the total weight of the stable pharmaceutical composition.

In one embodiment, the stable pharmaceutical composition consists essentially of laquinimod sodium, mannitol and magnesium stearate. In another embodiment, the stable pharmaceutical composition comprises, by total weight of the pharmaceutical composition, 0.21-0.35% of the pharmaceutically acceptable salt of laquinimod, 89.0-99.5% mannitol, and 0.5-2.0% magnesium stearate. In another embodiment, the stable pharmaceutical composition comprises, by total weight of the pharmaceutical composition, 0.15-0.35% of the pharmaceutically acceptable salt of laquinimod, 97.65-99.5% mannitol, and 0.5-2.0% magnesium stearate. In another embodiment, the stable pharmaceutical composition comprises, by total weight of the pharmaceutical composition, about 0.21% laquinimod sodium, about 98.80% mannitol and about 0.99% magnesium stearate. In another embodiment, the stable pharmaceutical composition comprises, by total weight of the pharmaceutical composition, 0.21% laquinimod sodium, 98.80% mannitol and 0.99% magnesium stearate. In another embodiment, the stable pharmaceutical composition comprises, by total weight of the pharmaceutical composition, about 0.64 mg laquinimod sodium, about 300 mg mannitol and about 3.0 mg magnesium stearate. In another embodiment, the stable pharmaceutical composition comprises, by total weight of the pharmaceutical composition, 0.64 mg laquinimod sodium, 300 mg mannitol and 3.0 mg magnesium stearate. In another embodiment, the stable pharmaceutical composition comprises, by total weight of the pharmaceutical composition, about 0.19% laquinimod sodium, about 98.94% mannitol and about 0.87% magnesium stearate. In another embodiment, the stable pharmaceutical composition comprises, by total weight of the pharmaceutical composition, 0.19% laquinimod sodium, 98.94% mannitol and 0.87% magnesium stearate.

In one embodiment, 10% or more of the total amount by volume of the laquinimod solid particles have a size of greater than 40 microns. In another embodiment, 50% or more of the total amount by volume of the laquinimod solid particles have a size of greater than 15 microns.

In one embodiment, the stable pharmaceutical composition is in the form of a tablet. In another embodiment, the stable pharmaceutical composition is in the form of a capsule.

The subject invention also provides a process for making a stable pharmaceutical composition comprising a therapeutically effective amount of laquinimod, an amount of a filler and an amount of a lubricant, wherein the pharmaceutical composition is free of an alkalizing agent or an oxidation reducing agent, said process comprising: a) obtaining the laquinimod, the lubricant and the filler; b) mixing the laquinimod, the lubricant and the filler from step a) to achieve a dry mix free of an alkalizing agent or an oxidation reducing agent; and c) compressing the dry mix of step b) to form a tablet.

In an embodiment of the present invention, the process comprises passing the lubricant through a mesh prior to step b). In another embodiment, the process comprises passing the filler through a mesh prior to step b).

The subject invention also provides a process for making a stable pharmaceutical composition comprising a therapeutically effective amount of laquinimod, an amount of a filler and an amount of a lubricant, wherein the pharmaceutical composition is free of an alkalizing agent or an oxidation reducing agent, said process comprising: a) obtaining the laquinimod, the lubricant and the filler; b) adding the filler to a mixer; c) dissolving laquinimod in water to form a laquinimod solution; d) adding the laquinimod solution of step c) to the mixer of step b); e) mixing the laquinimod solution and the mannitol to form a granulate; f) drying the granulate from step e) to form a dried granulate; g) screening the dried granulate of step f); h) milling the granulate resulting from step g) to form a milled granulate; i) adding the lubricant to the milled granulate of step h) to form a mixture; j) blending the mixture of step i) into a mixer to achieve a dry mix free of an alkalizing agent or an oxidation reducing agent; and k) filling the dry mix of step j) into a capsule or compressing the dry mix of step j) to form a tablet.

In an embodiment, the process comprises passing the lubricant through a mesh prior to step i). In another embodiment, the process comprises passing the filler through a mesh prior to step i).

The subject invention also provides a stable pharmaceutical composition comprising a therapeutically effective amount of laquinimod, an amount of a filler and an amount of a lubricant wherein the pharmaceutical composition is free of an alkalizing agent or an oxidation reducing agent, prepared by the processes described herein.

The subject invention also provides a sealed package comprising the stable pharmaceutical compositions described herein. In one embodiment, the sealed package further comprises a desiccant. In another embodiment, the desiccant is silica gel.

In one embodiment, the sealed package after storage at 40° C. and at a relative humidity (RH) of 75% for 2 months contains less than 0.5 wt % of a degradant of laquinimod.

The subject invention also provides a sealed package containing a stable pharmaceutical composition comprising a therapeutically effective amount of laquinimod, an amount of a filler and an amount of a lubricant, wherein the pharmaceutical composition is free of an alkalizing agent or an oxidation reducing agent, and wherein the sealed package has a moisture permeability of not more than 9.2 mg/day per liter.

The subject invention also provides a method for treating a subject afflicted with a form of multiple sclerosis comprising administering to the subject a stable pharmaceutical composition as described herein so as to thereby treat the subject.

The subject invention also provides a method for alleviating a symptom of multiple sclerosis in a subject afflicted with a form of multiple sclerosis comprising administering to the subject a stable pharmaceutical composition as described herein so as to thereby alleviate the symptom of multiple sclerosis in the subject.

The subject invention also provides for use of a stable pharmaceutical composition as described herein for treating a subject afflicted with a form of multiple sclerosis.

The subject invention also provides for use of a stable pharmaceutical composition as described herein for alleviating a symptom of multiple sclerosis in a subject afflicted with a form of multiple sclerosis.

For the foregoing embodiments, each embodiment disclosed herein is contemplated as being applicable to each of the other disclosed embodiments.

A dosage unit may comprise a single compound or mixtures of compounds thereof. A dosage unit can be prepared for oral dosage forms, such as tablets, capsules, pills, powders, and granules.

Laquinimod can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral administration. Laquinimod can be administered alone but is generally mixed with a pharmaceutically acceptable carrier, and co-administered in the form of a tablet or capsule, liposome, or as an agglomerated powder. Examples of suitable solid carriers include lactose, sucrose, gelatin and agar.

Capsule or tablets can be formulated and can be made easy to swallow or chew; other solid forms include granules and bulk powders. Tablets may contain suitable binders, lubricants, diluents, disintegrating agents (disintegrants), coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, dicalcium phosphate, calcium sulfate, mannitol, sorbitol, microcrystalline cellulose and the like. Suitable binders include starch, gelatin, natural sugars such as glucose or beta-lactose, corn starch, natural and synthetic gums such as acacia, tragacanth, or sodium alginate, povidone, carboxymethylcellulose, polyethylene glycol, waxes, and the like. Lubricants used in these dosage forms include sodium oleate, sodium stearate, sodium benzoate, sodium acetate, sodium chloride, stearic acid, sodium stearyl fumarate, talc and the like. Disintegrants include, without limitation, starch, methyl cellulose, agar, bentonite, xanthan gum, croscarmellose sodium, sodium starch glycolate and the like.

Specific examples of the techniques, pharmaceutically acceptable carriers and excipients that may be used to formulate oral dosage forms of the present invention are described, e.g., in U.S. Patent Application Publication No. 2005/0192315, PCT International Application Publication Nos. WO 2005/074899, WO 2007/047863, and WO/2007/146248, each of which is hereby incorporated by reference into this application.

General techniques and compositions for making dosage forms useful in the present invention are described in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol. 40 (Gilbert S. Banker, Christopher T. Rhodes, Eds.). These references in their entireties are hereby incorporated by reference into this application.

Terms

As used herein, and unless stated otherwise, each of the following terms shall have the definition set forth below.

As used herein, “laquinimod” means laquinimod acid or a pharmaceutically acceptable salt thereof.

A “salt” is salt of the instant compounds which have been modified by making acid or base salts of the compounds. The term “pharmaceutically acceptable salt” in this respect, refers to the relatively non-toxic, inorganic and organic acid or base addition salts of compounds of the present invention. A pharmaceutically acceptable salt of laquinimod as used in this application includes lithium, sodium, potassium, magnesium, calcium, manganese, copper, zinc, aluminum and iron. Salt formulations of laquinimod and the process for preparing the same are described, e.g., in U.S. Pat. No. 7,589,208 and PCT International Application Publication No. WO 2005/074899, which are hereby incorporated by reference into this application.

As used herein, “alkalizing agent” is used interchangeably with the term “alkaline-reacting component” or “alkaline agent” and refers to any pharmaceutically acceptable excipient which neutralizes protons in, and raises the pH of, the pharmaceutical composition in which it is used.

As used herein, “oxidation reducing agent” refers to a group of chemicals which includes an “antioxidant”, a “reduction agent” and a “chelating agent”.

As used herein, “antioxidant” refers to a compound selected from the group consisting of tocopherol, methionine, glutathione, tocotrienol, dimethyl glycine, betaine, butylated hydroxyanisole, butylated hydroxytoluene, turmerin, vitamin E, ascorbyl palmitate, tocopherol, deteroxime mesylate, methyl paraben, ethyl paraben, butylated hydroxyanisole, butylated hydroxytoluene, propyl gallate, sodium or potassium metabisulfite, sodium or potassium sulfite, alpha tocopherol or derivatives thereof, sodium ascorbate, disodium edentate, BHA (butylated hydroxyanisole), a pharmaceutically acceptable salt or ester of the mentioned compounds, and mixtures thereof.

The term “antioxidant” as used herein also refers to flavonoids such as those selected from the group of quercetin, morin, naringenin and hesperetin, taxifolin, afzelin, quercitrin, myricitrin, genistein, apigenin and biochanin A, flavone, flavopiridol, isoflavonoids such as the soy isoflavonoid, genistein, catechins such as the tea catechin epigallocatechin gallate, flavonol, epicatechin, hesperetin, chrysin, diosmin, hesperidin, luteolin, and rutin.

As used herein, “reduction agent” refers to a compound selected from the group consisting of thiol-containing compound, thioglycerol, mercaptoethanol, thioglycol, thiodiglycol, cysteine, thioglucose, dithiothreitol (DTT), dithio-bis-maleimidoethane (DTME), 2,6-di-tert-butyl-4-methylphenol (BHT), sodium dithionite, sodium bisulphite, formamidine sodium metabisulphite, and ammonium bisulphite.

As used herein, “chelating agent” refers to a compound selected from the group consisting of penicillamine, trientine, N,N′-diethyldithiocarbamate (DDC), 2,3,2′-tetraamine (2,3,2′-tet), neocuproine, N,N,N′,N′-tetrakis(2-pyridylmethyl)ethylenediamine (TPEN), 1,10-phenanthroline (PHE), tetraethylenepentamine, triethylenetetraamine and tris(2-carboxyethyl) phosphine (TCEP), ferrioxamine, CP94, EDTA, deferoxainine B (DFO) as the methanesulfonate salt (also known as desferrioxanilne B mesylate (DFOM)), des feral from Novartis (previously Ciba-Giegy), and apoferritin.

As used herein, a composition that is “free” of a chemical entity means that the composition contains, if at all, an amount of the chemical entity which cannot be avoided although the chemical entity is not part of the formulation and was not affirmatively added during any part of the manufacturing process. For example, a composition which is “free” of an alkalizing agent means that the alkalizing agent, if present at all, is a minority component of the composition by weight. Preferably, when a composition is “free” of a component, the composition comprises less than 0.1 wt %, 0.05 wt %, 0.02 wt %, or 0.01 wt % of the component.

As used herein, “about” in the context of a numerical value or range means ±10% of the numerical value or range recited or claimed.

An “amount” or “dose” of laquinimod as measured in milligrams refers to the milligrams of laquinimod acid present in a preparation, regardless of the form of the preparation.

The term “stable pharmaceutical composition” as used herein in connection with the composition according to the invention denotes a composition, which preserves the physical stability/integrity and/or chemical stability/integrity of the active pharmaceutical ingredient during storage. Furthermore, “stable pharmaceutical composition” is characterized by its level of degradation products not exceeding 5% at 40° C./75% RH after 6 months or 3% at 55° C./75% RH after two weeks, compared to their level in time zero.

As used herein, “treating” encompasses, e.g., inducing inhibition, regression, or stasis of a disease, disorder or condition, or ameliorating or alleviating a symptom of a disease, disorder or condition. “Ameliorating” or “alleviating” a condition or state as used herein shall mean to relieve or lessen the symptoms of that condition or state. “Inhibition” of disease progression or disease complication in a subject as used herein means preventing or reducing the disease progression and/or disease complication in the subject.

As used herein, “effective” as in an amount effective to achieve an end, i.e., “therapeutically effective amount”, means the quantity of a component that is sufficient to yield an indicated therapeutic response without undue adverse side effects (such as toxicity, irritation, or allergic response) commensurate with a reasonable benefit/risk ratio when used in the manner of this disclosure. For example, an amount effective to treat a subject afflicted with a form of multiple sclerosis. The specific effective amount will vary with such factors as the particular condition being treated, the physical condition of the patient, the type of mammal being treated, the duration of the treatment, the nature of concurrent therapy (if any), and the specific formulations employed and the structure of the compounds or its derivatives.

“Administering to the subject” means the giving of, dispensing of, or application of medicines, drugs, or remedies to a subject to relieve, cure, or reduce the symptoms associated with a condition, e.g., a pathological condition.

As used herein, “pharmaceutically acceptable carrier” refers to a carrier or excipient that is suitable for use with humans and/or animals without undue adverse side effects (such as toxicity, irritation, and allergic response) commensurate with a reasonable benefit/risk ratio. It can be a pharmaceutically acceptable solvent, suspending agent or vehicle, for delivering the instant compounds to the subject. “Pharmaceutically acceptable carrier” includes “fillers”, which fill out the size of a tablet or capsule, making it practical to produce and convenient for the consumer to use. By increasing the bulk volume, the fillers make it possible for the final product to have the proper volume for patient handling. “Pharmaceutically acceptable carrier” also includes “lubricants”, which prevent ingredients from clumping together and from sticking to the tablet punches or capsule filling machine. Lubricants also ensure that tablet formation and ejection can occur with low friction between the solid and die wall.

It is understood that where a parameter range is provided, all integers within that range, and tenths and hundredth thereof, are also provided by the invention. For example, “0.15-0.35%” includes 0.15%, 0.16%, 0.17% etc. up to 0.35%.

This invention will be better understood by reference to the Experimental Details which follow, but those skilled in the art will readily appreciate that the specific experiments detailed are only illustrative of the invention as described more fully in the claims which follow thereafter.

### EXPERIMENTAL DETAILS

### EXAMPLE 1

### Compatibility of Laquinimod Sodium with Fillers, with or without Addition of Water

In HDPE securitainers (canister) several binary blends were prepared containing laquinimod sodium and a filler (mannitol or lactose), with or without water, as presented in Table 1.

TABLE 1
Dry and wet compositions of laquinimod sodium with fillers

| Batch No. | Active Material | Excipient | Water |
|---|---|---|---|
| 1 | Laquinimod sodium 1 | Mannitol 300 mg (466.66) | − |
| 2 | Laquinimod sodium 1 | Mannitol 300 mg (466.66) | + |
| 3 | Laquinimod sodium 1 | Lactose monohydrate | − |
| | | 160 mg (248.88) | |
| 4 | Laquinimod sodium 1 | Lactose monohydrate | + |
| | | 160 mg (248.99) | |

Preparing Batches 1 and 3 (Dry) and Batches 2 and 4 (Wet)

The dry blend was prepared by placing 4.5 mg laquinimod sodium and 2.1 g mannitol or 1.12 g lactose monohydrate into a plastic securitainer (HDPE canister). The securitainers were closed with a polypropylene cap and were placed into a V type blender. Then they were mixed for 10 minutes to form Batch 1 and Batch 3.

The wet blend was prepared by placing 4.5 mg laquinimod sodium and 2.1 g mannitol or 1.12 g lactose monohydrate into a plastic securitainer (HDPE canister). The securitainers were closed with polypropylene cap and were placed into a V-blender. Then they were mixed for 10 minutes to form Batch 2 and Batch 4. The polypropylene cap was then opened, and 10 drops of water were added to each securitainer, and the content was mixed with a spatula to ensure wetting of the powder. The securitainers were closed again with the polypropylene caps.

All the blends were placed into stability chamber at 55° C. for two weeks.

After two weeks the blends were tested for Assay, Polar and Non Polar IDD. The results are presented in Tables 2 and 3.

TABLE 2
Results of the dry vs. wet compositions

| | | MCQ | | | RRT | RRT | RRT | RRT | |
|---|---|---|---|---|---|---|---|---|---|
| Batch No. | 5-HLAQ | & MCQCA | MCQME | MCQEE | ~0.19 | ~0.20 | ~0.22 | ~1.20 | Total |
| 1 | — | — | — | — | — | 0.08 | 0.04 | — | 0.12 |
| 2 | <0.05 | 0.40 | <0.02 | <0.02 | 0.11 | 0.08 | 0.05 | 0.24 | 0.88 |

TABLE 3
Results of the dry vs. wet compositions

| | | RRT | RRT | RRT | RRT | RRT | RRT | RRT | |
|---|---|---|---|---|---|---|---|---|---|
| Batch No. | 5-HLAQ | ~0.14 | ~0.17 | ~0.18 | ~0.21 | ~0.23~0.81 | ~1.17 | ~1.50 | Total |
| 3 | — | 0.01 | 0.03 | 0.01 | — | — | 0.05 | 0.03 | 0.13 |
| 4 | 0.05 | — | 0.09 | — | 0.09 | 0.14 | — | 0.03 | 0.40 |

No significant Non Polar Impurities were obtained in all compositions.

According to the results presented in Table 2 and Table 3, superior stability results were obtained in the dry blends (Batches 1 and 3), compared to the wet blends in both formulations.

In the laquinimod sodium—mannitol wet blend (Batch 2), a total of 0.88% Polar IDD was obtained while the dry blend (Batch 1), obtained a total of 0.12% Polar IDD.

In laquinimod sodium-lactose wet blend (Batch 4), a total of 0.40% Polar IDD was obtained while the dry blend (Batch 3) obtained a total of 0.13% Polar IDD.

In order to evaluate the differences between dry and wet manufacturing processes, a comparison was made between capsules prepared by a dry mix process vs. capsules prepared by wet granulation, with Pruv® (sodium stearyl fumarate) as a lubricant. The capsules were packed in 50 cc Duma® bottles with polypropylene cap (2 g silica gel inserted in cap). The compositions of the dry formulation (Batch 5) and the wet formulation (Batch 6), without alkalizing agent, are presented in Table 4.

TABLE 4
Dry mix and wet granulation formulations

Batch No.

| Composition (mg) | 5 | 6 |
|---|---|---|
| Laquinimod sodium | 0.64 | 0.64 |
| Mannitol | 300.00 | 340.00 |
| Pruv ® | 3.0 | 3.4 |
| Total | 303.64 | 344.04 |

Manufacturing Batches 5 and 6

Batch 5 (Dry)

Mannitol was screened using a 30 mesh sieve and inserted with laquinimod sodium into a V type blender. The mixture was then blended for 15 minutes. The lubricant (Pruv®) was screened using a 50 mesh sieve, added to the V type blender and blended for an additional 5 minutes.

304 mg of the final blend were then filled into size 1 white opaque gelatin capsules. The capsules were packed into 50 cc Duma® bottles with polypropylene cap (2 g silica gel inserted in cap).

The capsules were placed in a stability chamber at 40° C./75% RH for 3 months and tested for Assay, Dissolution, Polar and Non Polar IDD.

Batch 6 (Wet)

For the purpose of manufacturing Batch 6, mannitol was placed into a high shear mixer. Laquinimod sodium was dissolved in purified water and was added to the mannitol. The mannitol and the granulation solution were mixed in the high shear mixer to obtain the desired granulate.

The granulate obtained was dried in a Fluid Bed Dryer until a loss on drying (LOD) of not more than 0.5% was obtained. The dried granulate was milled using a 0.8 mm screen. The milled granulate was transferred to the V type blender.

Lubricant (PRUV®) was screened using a 50 mesh sieve, added to the V type blender and blended for an additional 5 minutes. 344 mg of the final blend were then filled into size 1 white opaque gelatin capsules. The capsules were packed into 50 cc Duma® bottles with polypropylene cap (2 g silica gel inserted in cap).

The capsules (Batches 5 and 6) were placed in a stability chamber at accelerated conditions for 3 months. The results for Polar IDDs are shown in Table 5.

TABLE 5

| | | | Any other impurities at RRT | | | | Total |
|---|---|---|---|---|---|---|---|
| | | MCQ + | ~0.15 | ~0.18 | ~0.20 | ~1.29 | Polar |
| Batch | Interval | MCQCA | ~0.17 | ~0.19 | ~0.28 | ~1.42 | IDD |

| | | | | | | |
|---|---|---|---|---|---|---|
| 5 (Dry mix, Pruv ®, | T₀ | <0.02 | | | | <0.05 |
| Duma ®) | 3 M | 0.06 | 0.13 | <0.02 | | 0.19 |
| 6 (Wet granulation, Pruv ®, | T₀ | <0.05 | <0.05 | <0.05 | — | <0.05 |
| Duma ®) | 2 M | 0.26 | 0.16 | <0.05 | — | 0.42 |
| | 3 M | 0.48 | 0.31 | 0.08 | 0.05 | 0.92 |

No significant Non Polar IDD was obtained in both granulates. Similar to compatibility results before, after 3 months at accelerated conditions, total Polar IDDs obtained in dry blend (Batch 5) is 0.19%, which is better than total Polar IDDs of 0.92% obtained in wet granulation (Batch 6).

### EXAMPLE 2

### Packaging Influence on Batches Manufactured Using a Wet Granulation Process

A comparison was made between stability results obtained at accelerated conditions of capsules manufactured using wet granulation process, which were packed in HDPE bottles with and without a desiccant (silica gel) (Batch 6 versus Batch 7). The proportional formulations tested are presented in Table 6.

TABLE 6

| | |
|---|---|
| Composition | Batch No. |

| (mg) | 6 (capsules) | 7 (capsules) |
|---|---|---|
| Laquinimod | 0.64 | 0.32 |
| Mannitol | 340.00 | 170.0 |
| Pruv ® | 3.4 | 1.6 |
| Total | 344.04 | 171.9 |

The manufacturing of Batch 6 was described in Example 1.

Manufacturing Batch 7

Mannitol was placed into a high shear mixer. Laquinimod sodium was dissolved in purified water and added to the mannitol. The mannitol and the granulation solution were mixed in the high shear mixer to obtain the desired granulate.

The granulate obtained was dried in a Fluid Bed Dryer until a loss on drying (LOD) of not more than 0.5% was obtained. The dried granulate was milled using a 0.8 mm screen. The milled granulate was transferred to the V type blender.

Lubricant (PRUV®) was screened using a 50 mesh sieve, added to the V type blender and blended for an additional 5 minutes.

The final blend was filled into orange opaque hard gelatin capsules, size 3 (weight: 171.9 mg/capsule) and the capsules were packed into 30 cc HDPE bottles with induction liner and polypropylene cap without silica gel.

The capsules were placed in stability chamber at accelerated conditions for 2 months. Results for Polar IDDs are presented in Table 7.

TABLE 7

| | | | Any other impurities at RRT | | | | | |
|---|---|---|---|---|---|---|---|---|
| | | MCQ + | ~0.15 | ~0.18 | ~0.20 | ~1.29 | | |
| Batch No. | Interval | MCQCA | ~0.17 | ~0.19 | ~0.29 | ~1.42 | Each | Total |
| Specification | | NMT: 0.5% | NMT: 0.5% | | | | | NMT 2.0% |
| 7 (Induction without | T₀ | <0.02 | — | — | — | — | — | 0.07 |
| Silica Gel) | 1 M | 0.79 | — | 0.27 | 0.07 | 0.13 | — | 1.26 |
| | 2 M | 1.74 | 0.05 | | 0.43 | | | 2.22 |
| 6 (Duma ®) | T₀ | <0.05 | — | <0.05 | <0.05 | — | — | <0.05 |
| | 1 M | 0.15 | — | 0.08 | <0.05 | <0.05 | — | 0.23 |
| | 2 M | 0.26 | — | 0.16 | <0.05 | — | — | 0.42 |
| | 3 M | 0.48 | — | 0.31 | 0.08 | 0.05 | — | 0.92 |

No significant Non Polar IDD was obtained in both packaging configurations.

After 2 months at accelerated conditions, high impurity levels were obtained in HDPE bottles without desiccant (Batch 7) with total Polar IDDs 2.22% vs. 0.42% in Duma® bottle with polypropylene cap and 2 g silica gel inserted in cap (Batch 6).

### EXAMPLE 3

### Lubricant Influence in Dry Blend

Based on the results obtained in compatibility between laquinimod and mannitol in dry blend (Batch 1), two different lubricants were added to this combination without addition of alkalizing agent. A dry blend (Batch 5) was prepared from laquinimod, mannitol and Pruv® (Sodium Stearyl Fumarate) and other dry blend (Batch 8) was prepared from laquinimod, mannitol and magnesium stearate as presented in Table 8.

TABLE 8
Dry blend formulations with different Lubricant

Batch No.

| Composition | 5 | 8 |
|---|---|---|
| Laquinimod | 0.64 | 0.64 |
| Mannitol USP/BP | 300.00 | 300.00 |
| Mg. Stearate | — | 3.0 |
| Pruv ® | 3.0 | — |
| Total | 303.64 | 303.64 |

Manufacturing Batch 8

Mannitol was passed through sieve 30 mesh and then blend with laquinimod into y-cone for 15 minutes. The lubricant (Pruv®/magnesium stearate) was passed through sieve 50 mesh and was added to the blend of laquinimod with mannitol, then continued blending for 5 minutes.

The blend was filled into size 1, white opaque gelatin capsules (weight: 303.64 mg/capsule). The capsules were packed into 50 cc Duma® bottles with polypropylene cap (2 g silica gel inserted in cap).

The capsules were placed in stability chamber at 40° C./75% RH for 6 months and tested for Assay, Dissolution, Polar and Non Polar IDD. The results are shown in Table 9 (Polar IDD (%) at 40° C./75% RH).

TABLE 9
The influence of different lubricant

| | | | Any other impurities at RRT | | | | Total | | |
|---|---|---|---|---|---|---|---|---|---|
| | | MCQ + | ~0.15 | ~0.18 | ~0.20 | ~1.29 | Polar | DIS | |
| Batch No. | Interval | MCQCA | ~0.17 | ~0.19 | ~0.28 | ~1.42 | IDD | 30 min | Water |

| | | | | | | | |
|---|---|---|---|---|---|---|---|
| 5 (Pruv ® | T₀ | <0.02 | | | <0.05 | 93 | 0.08 |
| Duma ®) | 3 M | 0.06 | 0.13 | <0.02 | 0.19 | 95 | 0.04 |
| | 6 M | 0.25 | 0.51 | 0.15 | 0.91 | 96 | 0.05 |
| 8 | T₀ | <0.03 | | | <0.05 | 97 | 0.08 |
| (Magnesium | 3 M | <0.03 | <0.02 | <0.02 | <0.05 | 99 | 0.05 |
| stearate, | 6 M | <0.03 | 0.05 | | <0.05 | 99 | 0.06 |
| Duma ®) | | | | | | | |

No significant non polar impurities were obtained in both formulations.

After 6 months at accelerated conditions, low impurities (Total Polar IDD: <0.05%) were obtained in capsules with magnesium stearate as lubricant (batch 8). In capsules with Pruv® as lubricant (batch 5), the impurities were higher than in capsules with magnesium stearate (total Polar IDD: 0.91%) but the results were still within specifications (NMT 2%). Sum of MCQ+MCQCA obtained was 0.25%, which is still within specifications (NMT 0.5%).

### EXAMPLE 4

### Tablet Formulations Using Different Fillers

Two dry blends were prepared and tablets were pressed. The first blend (Batch 9) is a combination of laquinimod and Mannitol Partek M200 as filler and the second blend (Batch 10) is a combination of laquinimod and lactose spray dried as filler. In both blends magnesium stearate was used as lubricant. The two blends without alkalizing agent are presented in Table 10.

TABLE 10
Tablets formulation with different fillers

Batch No.

| Composition | 9 (tablets.) | 10 (tablets) |
|---|---|---|
| Laquinimod | 0.64 | 0.64 |
| Mannitol (Partek M200 in tablets) | 300.36 | — |
| Lactose SD | — | 300.36 |
| Magnesium Stearate | 3.00 | 1.50 |
| Total | 304.00 | 302.50 |

Manufacturing Batches 9 and 10

Mannitol Partek or lactose spray dried and laquinimod sodium were mixed into Y-cone for 10 minutes. Magnesium stearate was passed through mesh 50 and was added to the Y-cone and continued mixing for 5 minutes. Tablets were pressed by Sviac press machine. The tablets were packed in 50 cc HDPE Duma® bottles with polypropylene cap (2 g silica gel inserted in cap) and placed in stability chamber at 40° C./75% RH for 6 months. The results are presented in Table 11 (Polar IDD (%) at 40° C./75% RH).

TABLE 11
The influence of different fillers in tablets formulation

| | | | Any other impurities at RRT | | | | | |
|---|---|---|---|---|---|---|---|---|
| | | MCQ + | ~0.15 | ~0.18 | ~0.20 | ~1.29 | | |
| Batch No. | Interval | MCQCA | ~0.17 | ~0.19 | ~0.29 | ~1.42 | Each | Total |
| Specification | | NMT: 0.5% | NMT: 0.5% | | | | | NMT: 2.0% |
| 9 (Mannitol, | T₀ | <0.03 | — | — | — | — | <0.02 | <0.05 |
| Duma ®) | 3 M | <0.03 | — | — | — | — | <0.02 | <0.05 |
| | 6 M | <0.03 | — | — | — | — | <0.02 | <0.05 |
| 10 (Lactose, | T₀ | <0.03 | — | — | — | — | — | <0.1 |
| Duma ®) | 3 M | 0.07 | — | — | — | — | — | 0.07 |
| | 6 M | 0.18 | | 0.05 | 0.07 | | | 0.3 |

No significant Non Polar IDD was obtained.

The results obtained after 6 months were satisfactory in both formulations: Total Polar IDD in tablets with mannitol was <0.05% and 0.3% in tablets with lactose.

### EXAMPLE 5

### Comparison Between Capsules and Tablets Without Alkalizing Agent

Batches 8 and 9 were manufactured according to previously described procedure in order to compare capsules and tablets without alkalizing agent. The two blends without alkalizing agent are presented in Table 12.

TABLE 12
Composition of capsules and tablets

Batch No.

| Composition | 8 (capsules) | 9 (tablets) |
|---|---|---|
| Laquinimod | 0.64 | 0.64 |
| Mannitol (Partek M200 in tablets) | 300.00 | 300.36 |
| Magnesium Stearate | 3.0 | 3.00 |
| Total | 303.64 | 304.00 |

A comparison between capsules and tablets, formulated from dry blend laquinimod sodium, mannitol and magnesium stearate as lubricant (without an alkalizing agent) provided, in both formulations, good results (table 13; Polar IDD (%) at 40° C./75% RH).

TABLE 13
Capsules vs. tablets

| | | | Any other impurities at RRT | | | | | |
|---|---|---|---|---|---|---|---|---|
| | | MCQ + | ~0.15 | ~0.18 | ~0.20 | ~1.29 | | |
| Batch No. | Interval | MCQCA | ~0.17 | ~0.19 | ~0.28 | ~1.42 | Each | Total |
| Specification | | NMT: 0.5% | NMT: 0.5% | | | | | NMT 2.0% |
| 8 (Capsules, | T₀ | <0.03 | | | | | | <0.05 |
| Dry blend) | 3 M | <0.03 | | <0.02 | <0.02 | | | <0.05 |
| | 6 M | <0.03 | | 0.05 | | | | <0.05 |
| 9 (Tablets, | T₀ | <0.03 | | | | | <0.02 | <0.05 |
| Dry blend) | 3 M | <0.03 | | | | | <0.02 | <0.05 |
| | 6 M | <0.03 | | | | | <0.02 | <0.05 |

Batches 8 and 9, which were packaged in DUMA® bottles (containing 2 g desiccant) and did not contain an alkalizing agent, had shown that at accelerated conditions, for up to 6 months, no impurities were formed. Two additional batches (Batches 16 and 17) were manufactured to assess the effect of a disintegrant (croscarmellose sodium) on the dissolution rate of the tablets. Stability of the batches at 55° C./75% RH and at accelerated conditions was tested. The batches were manufactured using a dry granulation process with milling. Both batches were then packaged in LOG 60 ml bottles, with or without 1 g silica gel (Batches 16A; 16B; 17A; 17B) are described in Table 14.

TABLE 14
Formulation without alkalizing agent, with or without
disintegrant and with or without desiccant

Batch Number

| Composition | 16A | 16B | 17A | 17B |
|---|---|---|---|---|
| Mannitol | + | + | + | + |
| Laquinimod Sodium | + | + | + | + |
| Magnesium Stearate | + | + | + | + |
| Croscarmellose Sodium | − | − | + | + |
| (Ac-Di-Sol) | | | | |
| Silica gel 1 g | + | − | + | − |

Stability results of these batches at 55° C./75% RH and 40° C./75% RH, for up to 1 month did not show a major increase in impurities. The presence of 1 g desiccant was shown to have a good impact on stability by decreasing the level of impurities (Table 15; Polar IDDs (%) at 55° C./75% RH and at 40° C./75% RH). The dissolution results for both batches showed that satisfactory dissolution can be achieved even without a disintegrant.

TABLE 15
Stability and impurity study with (A) or without (B) 1 g desiccant.

| | | | 1-WEEK | 2-WEEK | 2-WEEK- | 1-MONTH | 1-MONTH |
|---|---|---|---|---|---|---|---|
| | | | (55° C./ | (55° C./ | (40° C./ | (55° C./ | (40° C./ |
| Batch | Component | Time 0 | 75% RH) | 75% RH) | 75% RH) | 75% RH) | 75% RH) |
| 16A | Assay avg. (%) | 99 | 98 | 97 | 99 | 97 | 96 |
| | Impurities | | 0.22% | 0.30% | 0.11% | 0.31% | <0.05% |
| | (total) | | | | | | |
| | WATER (%) | 0.2 | 0.1 | 0.1 | 0.1 | 0.1 | 0.1 |
| | Dissolution | 99 | 100 | 99 | 99 | 99 | 99 |
| | (15 min) | | | | | | |
| 16B | Assay avg. (%) | 99 | 98 | 96 | 99 | 96 | 97 |
| | Impurities | | 0.49% | 0.49% | <0.05% | 0.61% | 0.08% |
| | (total) | | | | | | |
| | WATER (%) | 0.2 | 0.2 | 0.2 | 0.2 | 0.1 | 0.1 |
| | Dissolution | 99 | | 97 | 101 | 99 | 100 |
| | (15 min) | | | | | | |
| 17A | Assay avg. (%) | 100 | 99 | 97 | 100 | 96 | 98 |
| | Impurities | | 0.28% | 0.47% | <0.05% | 0.73% | <0.05% |
| | (total) | | | | | | |
| | WATER (%) | 0.3 | 0.2 | 0.3 | 0.2 | 0.2 | 0.2 |
| | Dissolution | 100 | 99 | 102 | 102 | 99 | 100 |
| | (15 min) | | | | | | |
| 17B | Assay avg. (%) | 100 | 99 | 94 | 100 | | 97 |
| | Impurities | | 0.70% | 2.53% | <0.05% | | <0.05% |
| | (total) | | | | | | |
| | WATER (%) | 0.3 | 0.3 | 0.3 | 0.2 | | 0.3 |
| | Dissolution | 100 | 100 | 100 | 99 | | 101 |
| | (15 min) | | | | | | |

### EXAMPLE 6

### General Excipient Compatibility Study

Several excipient compatibility studies were performed. Due to the fact that tablet dosage form may require different excipients or grades compared to capsules, additional excipient compatibility study was performed. Different excipients were chosen for this study so they would be able to support wet and dry processes. Table 16 shows all the materials that were assessed during the excipients compatibility study.

TABLE 16
Excipient compatibility study list

| | Excipient:API | | |
|---|---|---|---|
| Material | Ratio | Material/grade | Main Function |
| Isomalt | 100:1 | GalenIQ 801 | Filler |
| Pregelatinised starch | 100:1 | Starch 1500 | Multifunctional |
| Lactose-starch | 100:1 | StarLac | Filler |
| Maltodextrin | 100:1 | Lycatab DSH | Multifunctional |
| Lactose anhydrous | 100:1 | Supertab 21AN | Filler |
| Hydroxypropyl | 8:1 | Klucel EXF | Dry binder |
| cellulose | | | |
| Hydroxypropyl | 8:1 | Methocel E5 | Wet binder |
| methylcellulose | | Premium | |
| PVP/VA 64 | 8:1 | Kollidon VA64 | Wet binder |
| Crospovidone | 10:1 | Kollidon CL | Disintegrant |
| Sodium starch | 10:1 | Explotab | Disintegrant |
| glycolate | | | |
| Sodium carbonate | 8:1 | Merck Darmstadt | pH modifier |
| Sodium citrate | 15:1 | Merck Darmstadt | pH modifier |
| dihydrate | | | |
| Magnesium oxide | 8:1 | Merck Darmstadt | pH modifier |
| Magnesium stearate | 3:1 | Mallinckrodt | Lubricant |

All excipients were mixed with the API at ratios that are recommended to be used in a typical formulation, with or without addition of water, and placed in 55° C./75% RH for up to 4 weeks. In addition, 2 potential formulations with filler, binder, disintegrant, API and a lubricant were compressed into tablets and placed under the same conditions.

All batches were manufactured using the excipients evaluated in the past or in the current compatibility study, as listed in Tables 17 and 18, and varied in the percentage of each excipient and process parameters.

TABLE 17
Potential excipients and their percentage in the
formulation

| Chemical name | Grade | Function | Percentage |
|---|---|---|---|
| Isomalt | GalenIQ 721 | Filler/diluent | 0, 50, 100 |
| Co processed lactose/ | StarLac | Filler/diluent | 0, 50, 100 |
| starch | | | |
| Croscarmellose sodium | AC-DI-SOL | Disintegrant | 5 |
| Magnesium stearate | LIGAMED MF-2-V | Lubricant | 1 |

TABLE 18
Potential excipients and their percentage in the
formulation

| Chemical name | Grade | Function | Percentage |
|---|---|---|---|
| Isomalt | GalenIQ 801 | Filler/diluent | 0, 50, 100 |
| Mannitol | Pearlitol 200SD | Filler/diluent | 0, 50, 100 |
| Croscarmellose | AC-DI-SOL | Disintegrant | 5 |
| sodium | | | |
| Maltodextrin | Lycatab DSH | Wet binder | 10 |
| Sodium carbonate | Merck EMPROVE | Alkalizing agent | 0, 2.5, 5 |
| anhydrous | Ph. Eur, BP, NF | | |
| Magnesium | Merck EMPROVE | Alkalizing agent | 0, 2.5, 5 |
| oxide - heavy | Ph. Eur, BP, NF | | |
| Magnesium stearate | LIGAMED MF-2-V | Lubricant | 1 |

A total of 21 batches were manufactured at this stage, which are divided into 4 processes for evaluation as of the following:

(1) High shear dry mix—6 batches.

(2) Geometrical bin blending—5 batches.

(3) High shear wet granulation—4 batches.

(4) Top spray granulation—6 batches.

Results obtained from dry and wet batches which did not contain an alkalizing agent, at 55° C./75% RH, showed a major decrease in assay and in impurities (Table 19—Polar IDD) compared to Batch 16A (Table 14 and Table 15, formulated with mannitol as filler and without alkalizing agent) at the same conditions.

TABLE 19
Stability of various formulations at turbo conditions

| | | | | Water | | | | Total | Dissolution | |
|---|---|---|---|---|---|---|---|---|---|---|
| | | | | Content | | Assay | | Impurities | (NLT 85% | |
| | | | | (info only) | | (95-105%) | | (NMT | after 30 | |
| | | Appearance | | (%) | | (%) | | 2.0%) (%) | mins) (%) | |
| | | After 2 Weeks | | | T = 2 | | T = 2 | T = 2 | 30 | |
| Batch No. | Formulation | 55/75 | 40/75 | T = 0 | weeks | T = 0 | weeks | weeks | mins | ∞ |
| 11 | Dry blend, | White | White | 3.8 | 4.2 | 101 | 89 | 3.1 | 86 | 86 |
| | 50/50 | | | | | | | | | |
| | Isomalt/ | | | | | | | | | |
| | Starlac | | | | | | | | | |
| 12 | Dry blend, | White | White | 2.6 | 2.8 | 95 | 77 | 6.7 | 75 | 75 |
| | Isomalt | | | | | | | | | |
| 13 | Dry blend, | Slight brown | White | 5.2 | 5.2 | 97 | 93 | 1.6 | 92 | 92 |
| | Starlac | discoloration | | | | | | | | |
| | | on some | | | | | | | | |
| | | tablets | | | | | | | | |
| 14 | Top spray | White | White | 2.3 | 2.5 | 95 | 83 | 5.6 | 79 | 80 |
| | granulation, | | | | | | | | | |
| | 50/50 | | | | | | | | | |
| | Isomalt/ | | | | | | | | | |
| | mannitol, | | | | | | | | | |
| | no | | | | | | | | | |
| | alkalizing | | | | | | | | | |
| | agent | | | | | | | | | |
| 15 | Dry Blend, | White | White | 5.5 | 5.5 | 95 | Pending | 5.5 | 88 | 89 |
| | Isomalt/ | | | | | | | | | |
| | mannitol | | | | | | | | | |

### REFERENCES

1. PCT International Application Publication No. WO 2005/074899, published Aug. 18, 2005 (ACTIVE BIOTECH AB).
2. PCT International Application Publication No. WO 2007/047863, published Apr. 26, 2007 (TEVA PHARMACEUTICAL INDUSTRIES, LTD.).
3. PCT International Application Publication No. WO/2007/146248, published Dec. 21, 2007 (TEVA PHARMACEUTICAL INDUSTRIES, LTD.).
4. Polman et al., (2005) “Treatment with laquinimod reduces development of active MRI lesions in relapsing MS”, Neurology. 64:987-991.
5. Sandberg-Wollheim et al., (2005) “48-week open safety study with high-dose oral laquinimod in patients”, Mult Scler. 11:S154.
6. U.S. Patent Application Publication No. 2005/0192315, published Sep. 1, 2005 (Jansson et al.).
7. U.S. Pat. No. 6,077,851, issued Jun. 20, 2000 to Anders Bjork et al.
8. U.S. Pat. No. 6,875,869, issued Apr. 5, 2005 to Karl Jansson.
9. U.S. Pat. No. 7,589,208, issued Sep. 15, 2009 to Jansson et al.
10. U.S. Pat. No. 7,884,208, issued Feb. 8, 2011 to Anton Frenkel et al.
11. U.S. Pat. No. 7,989,473, issued Aug. 2, 2011 to Shulamit Patashnik et al.
12. U.S. Pat. No. 8,178,127, issued May 15, 2012 to Muhammad Safadi et al.

1. A stable pharmaceutical composition comprising:
a) a therapeutically effective amount of laquinimod,
b) an amount of a filler, and
c) an amount of a lubricant,
wherein the stable pharmaceutical compositions free of an alkalizing agent or an oxidation reducing agent.

2. The stable pharmaceutical composition of claim 1 in a solid form composition.

3. The stable pharmaceutical composition of claim 1 or 2, which is free of an alkalizing agent and which is free of an oxidation reducing agent.

4. The stable pharmaceutical composition of claim 1, wherein the moisture content of the stable pharmaceutical composition is no more than 4%, less than 1.5% wt H₂O or less than 0.5% at H₂O.

5. (canceled)

6. (canceled)

7. The stable pharmaceutical composition of claim 1, wherein the total amount of non-polar impurities in the composition is less than 0.5 wt % relative to the amount of laquinimod.

8. The stable pharmaceutical composition of claim 1, wherein the filler, the lubricant and/or present in the composition as solid particles.

9. The stable pharmaceutical composition of claim 8, wherein the filler is lactose, lactose monohydrate, starch, isomalt, mannitol, sodium starch glycolate, sorbitol, lactose spray dried, lactose anhydrous, or a combination thereof, preferably the filler is mannitol or lactose monohydrate.

10. (canceled)

11. (canceled)

12. The stable pharmaceutical composition of claim 8, wherein the lubricant is magnesium stearate or sodium stearyl fumarate.

13. The stable pharmaceutical composition of claim 1, wherein the stable pharmaceutical composition is free of disintegrant and/or free of croscarmellose sodium.

14. (canceled)

15. The stable pharmaceutical composition of claim 1, wherein laquinimod is a pharmaceutically acceptable salt of laquinimod, which pharmaceutically acceptable salt is lithium salt, sodium salt or calcium salt, preferably the pharmaceutically acceptable salt of laquinimod is laquinimod sodium.

16. (canceled)

17. (canceled)

18. The stable pharmaceutical composition of claim 1, wherein the therapeutically effective amount of laquinimod is 0.25 mg-1.5 mg, preferably the theraceutically effective amount of laquinimod is 0.5 mg, 0.6 mg, 1.0 mg or 1.2 mg.

19-22. (canceled)

23. The stable pharmaceutical composition of claim 1, wherein the lubricant is between 0.5-2.0% of the total weight of the stable pharmaceutical composition and/or wherein the filler is between 89.0-99.5% of the total weight of the stable pharmaceutical composition.

24. (canceled)

25. The stable pharmaceutical composition of claim 1, consisting essentially of laquinimod sodium, mannitol and magnesium stearate.

26. The stable pharmaceutical composition of claim 25, comprising, by total weight of the pharmaceutical composition,
a) 0.21-0.35% of the pharmaceutically acceptable salt of laquinimod, 89.0-99.5% mannitol, and 0.5-2.0% magnesium stearate; or
b) 0.15-0.35% of the pharmaceutically acceptable salt of laquinimod, 97.65-99.5% mannitol, and 0.5-2.0% magnesium stearate; or
c) about 0.21% laquinimod sodium, about 98.80% mannitol and about 0.99% magnesium stearate; or
d) 0.21% laquinimod sodium, 98.80% mannitol and 0.99% magnesium stearate; or
e) about 0.64 mg laquinimod sodium, about 300 mg mannitol and 3.0 mg about magnesium stearate; or
f) 0.64 mg laquinimod sodium, 300 mg mannitol and 3.0 mg magnesium stearate; or
g) about 0.19% laquinimod sodium, about 98.94% mannitol and about 0.87% magnesium stearate; or
h) 0.19% laquinimod sodium, 99.94% mannitol and 0.87% magnesium stearate.

27-33. (canceled)

34. The stable pharmaceutical composition of claim 8, wherein 10% or more of the total amount by volume of the laquinimod solid particles have a size of greater than 40 microns and/or wherein 50% or more of the total amount by volume of the laquinimod solid particles have a size of greater than 15 microns.

35. (canceled)

36. The stable pharmaceutical composition of claim 1, in the form of a tablet or a capsule.

37. (canceled)

38. A process for making a stable pharmaceutical composition comprising a therapeutically effective amount of laquinimod, an amount of a filler and an amount of a lubricant, wherein the pharmaceutical composition is free of an alkalizing agent or an oxidation reducing agent, said process comprising:
a) obtaining the laquinimod, the lubricant and the filler;
b) mixing the laquinimod, the lubricant and the filler from step a) to achieve a dry mix free of an alkalizing agent or an oxidation reducing agent; and
c) compressing the dry mix of step b) to form a tablet.

39-43. (canceled)

44. A stable pharmaceutical composition comprising a therapeutically effective amount of laquinimod, an amount of a filler and an amount of a lubricant wherein the pharmaceutical composition is free of an alkalizing agent or an oxidation reducing agent, prepared by the process of claim 38.

45. A sealed package comprising the stable pharmaceutical composition of claim 1 or containing a stable pharmaceutical composition comprising a therapeutically effective amount of laquinimod, an amount of a filler and an amount of a lubricant, wherein the pharmaceutical composition is free of an alkalizing agent or an oxidation reducing agent, and wherein the sealed package has a moisture permeability of not more than 9.2 mg/day per liter.

46-49. (canceled)

50. A method for treating or for alleviating a symptom of multiple sclerosis in a subject afflicted with a form of multiple sclerosis comprising administering to the subject the stable pharmaceutical composition of claim 1 so as to thereby treat or alleviate the symptom of multiple sclerosis in the subject.

51-53. (canceled)
